(19) United States
(12) Patent Application Publication
(10) Pub. No.: US 2004/0018244 A1
(43) Pub. Date: Jan. 29, 2004

(54) Lotion compositions

(75) Inventor: Catherine A. Piterski, River Vale, NJ (US)

Correspondence Address: Charles N.J. Ruggiero, Esq., Ohlandt, Greeley, Ruggiero & Perle, L.L.P., One Landmark Square, 10th Floor, Stamford, CT, 06901-2682, US

(73) Assignee: Playtex Products, Inc.

(21) Appl. No.: 10/387,122

(22) Filed: Mar. 12, 2003

Related U.S. Application Data
(60) Provisional application No. 60/363,632, filed on Mar. 12, 2002.

(51) Int. Cl. A61K 35/78; A61K 35/20

(52) U.S. Cl. 424/535; 424/745; 424/764

(57) ABSTRACT

There is provided a moisturizing lotion composition for use on the skin. The composition comprises one or more milk protein compounds and one or more essential oils. The one or more milk protein compounds provide enhanced moisturizing to the skin while the one or more essential oils provide a calming effect to the user of the lotion.

This application claims priority from U.S. Provisional Patent Application Serial No. 60/363,632, filed on Mar. 12, 2002.

### BACKGROUND OF THE INVENTION

1. Field of the Invention

The present invention relates to a lotion compositions. More particularly, the present invention relates to a lotion with one or more milk proteins for enhanced skin moisturizing and one or more essential oils for aromatherapy.

2. Description of the Prior Art

The favorable effect of milk or products derived from milk on cosmetic compositions, such as lotions for the skin, hair lotions, shampoos, softening creams, and soaps, is well known. Numerous compositions containing such ingredients have been described in the state of the art.

U.S. Pat. No. 3,959,491 discloses cosmetic creams and lotions containing sterilized milk and milk cream. To improve the preservation of such products in storage, a saturated fatty acid, triglyceride esters of saturated fatty acid, stabilizing aliphatic diol, sequestering agent and biocidal agent, in particular enzyme denaturant and bactericide, are incorporated into the cosmetic compositions. The cosmetics disclosed also contain the usual constituents of cosmetics, including a perfume, opacifier and others.

U.S. Pat. No. 4,460,571 discloses a cosmetic composition, including a lotion, with whole or powdered milk, which particularly lends itself to topodermic and capillary uses.

GB 2,056,854 discloses cosmetics, in particular a lotion and shampoo, containing, apart from the normal ingredients inherent in such products, very high proportions of milk (40-80%) and, as a stabilizing agent, propylene glycol.

GB 2,037,160 discloses a lyophilized product derived from milk having advantageous cosmetic properties, in particular for moisturizing and softening of the skin along with its regeneration. The lyophilized product is used, in a mixture, with a cosmetic composition to improve their properties.

DE 2,623,250 discloses cosmetic compositions, such as, shampoo, beauty lotion, shaving cream, and cream for the skin, containing milk. All of these products have in common, apart from milk and water, a surfactant substance, such as alkyl-sulfonate, sulfonated fatty acid, and the salts of tertiary amines, etc. The milk may be present in the form of whole milk, low fat milk, acidified milk, skimmed milk, yogurt, cream, etc. in aqueous form or in a powder. Furthermore, the compositions described may also contain, depending on the use for which they are intended, other ingredients such as lanolin, perfume, glycerin and other similar products.

DE 2,650,560 discloses cosmetic compositions containing milk in the form of yogurt. This milk is usable in the fresh state or in a powder. Among such cosmetic or cosmetic compositions, this application discloses liquid and solid products, in particular a beauty milk, cream, and aerosol for foam. These compositions contain, apart from yogurt, a preservative, antioxidant, chelator, and protease inhibitor.

U.S. Pat. No. 5,053,219 discloses cosmetic products containing a milk constituent, such as natural milk proteins in the form of micelles formed by the addition of calcium to an aqueous solution of casein. These micelles give the product light reflection properties analogous to those produced by the addition of a natural milk.

U.S. Pat. No. 5,314,873 discloses milk protein hydrolyzates and compositions for use as hair and skin treating agent. The milk protein hydrolyzate includes a mixture of peptide and free amino acid. The milk protein hydrolyzate is obtained by enzymatic hydrolysis of milk protein.

Aromatherapy and its various uses has been described in the prior art. U.S. Pat. No. 5,888,984 discloses a topical pharmaceutical composition of complex carbohydrate and essential oil. The composition reduces inflammation, assists in wound healing, protects against bruising, relieves itching, relieves pain and swelling and treats topical bacterial infections, such as acne.

U.S. Pat. No. 6,280,751 discloses medicinal and cosmetic compositions having essential oils in combination with herbs and/or spices. The compositions include topical compositions.

What is not appreciated by the prior art is a lotion composition that simultaneously moisturizes the skin while promoting calming and healing of the user by aromatherapy. The present lotion composition overcomes this deficiency by providing a moisturizing lotion composition with one or more milk protein compounds and one or more essential oils.

### SUMMARY OF THE INVENTION

It is an object of the present invention to provide a lotion composition.

It is another object of the present invention to provide such a lotion that has one or more milk protein compounds for enhanced skin moisturizing.

It is still another object of the present invention to provide such a lotion with one or more essential oils.

It is a further object of the present invention to provide such a lotion composition that simultaneously moisturizes skin while providing aromatherapy to the user.

These and other objects and advantages of the present invention are achieved by a moisturizing lotion composition according to the present invention. The composition has one or more milk proteins and one or more essential oils. The one or more milk protein compounds provide enhanced moisturizing to the skin, while the one or more essential oils promote a calming effect to the user of the lotion.

The present invention also provides a method of simultaneously moisturizing skin, while promoting a calming effect on the user through aromatherapy. The method comprises applying a lotion composition having one or more milk protein compounds and one or more essential oils to the skin.

### DETAILED DESCRIPTION OF THE INVENTION

The present invention is a lotion composition for moisturizing the skin. The lotion composition includes at least one milk protein compound to enhance moisturizing of the skin. The lotion composition has advantages over other commercially available baby lotions due to the inclusion of at least one essential oil in the lotion. The at least one essential oil is believed to provide a calming and/or healing effect to the user, especially an infant or toddler, of the lotion through aromatherapy.

The at least one milk protein compound in the composition of the present invention has been found to enhance the moisturizing of the skin. Suitable milk protein compounds for use in the present invention include, for example, hydrolyzed milk protein, hydrolyzed milk protein derivative, or any combinations thereof. Suitable hydrolyzed milk protein derivatives include, for example, palmitoyl derivative and quaternary ammonium salt derivative. Preferably, the milk protein used in the present lotion composition is a hydrolyzed milk protein sold under the tradename Hydrolactin 2500® by Croda, Inc.

To achieve enhanced moisturizing properties, the one or more milk protein compounds are present in the lotion composition in an amount about 0.001 weight percent (wt. %) to about 1 wt. %. Preferably, the one or more milk protein compounds are present in an amount about 0.01 wt. % to about 0.5 wt. %.

To promote a calming effect on the user of the lotion composition of the present invention, one or more essential oils are present in the lotion composition. It is believed that the one or more essential oils provide a calming effect to the user of the lotion by way of aromatherapy.

Aromatherapy has been used for centuries as a way to promote physical and mental well being. Essential oils extracted from flowers, herbs, spices, woods, and fibers, when inhaled, are believed to have subtle effects on a person's mind and emotions. In addition, it is believed that when the essential oils penetrate the skin and enter the bloodstream and immune system, they work in a physiological manner to promote healing.

Essential oils are highly scented droplets found in minute quantities in the flowers, stems, leaves, roots and barks of aromatic plants. They are highly fluid and exceptionally volatile and potent. Because of their high potency, very small amounts of essential oil are needed to experience their benefits.

Essential oils are complex mixtures of different organic molecules, such as terpenes, alcohols, esters, aldehydes, ketones and phenols. It is believed that it is the interaction between each and every component and/or molecule that gives an essential oil its particular character and unique therapeutic properties. Therefore, the use of one or more essential oils in the present lotion composition not only provides a calming effect to the infant, it may also provide beneficial healing effects.

Suitable essential oils for use in the present lotion composition include, for example, Abies Sibirica Oil, Amyris Balsamifera Oil, Anise (Illicium Verum) Oil, Balm Mint (Melissa Officinalis) Oil, Basil (Ocimum Basilicum) Oil, Bay (Pimenta Acris) Oil, Bee Balm (Monarda Didyma) Oil, Bergamot (Citrus Aurantium Bergamia) Oil, Birch (Betula Aba) Oil, Bitter Orange (Citrus Aurantium Amara) Oil, Cabbage Rose (Rosa Centifolia) Oil, Calendula Officinalis Oil, California Nutmeg (Torreya Californica) Oil, Camellia Sinensis Oil, Capsicum Frutescers Oleoresin, Caraway (Carum Carvi) Oil, Cardamon (Elettaria Cardamomum) Oil, Cedarwood (Cedrus Atlantica) Oil, Chamaecyparis Obtusa Oil, Chamomile (Anthemis Nobilis) Oil, Cinnamon (Cinnamomum Cassia) Oil, Citronella (Cymbopogon Nardus) Oil, Clary (Salvia Sclarea) Oil, Clove (Eugenia Caryophyllus) Oil, Cloveleaf (Eugenia Caryophyllus) Oil, Coriander (Coriandrum Sativum) Oil, Coriander (Coriandrum Sativum) Seed Oil, Cyperus Esculentus Oil, Cypress (Cupressus Sempervirens) Oil, Eucalyptus Citriodora Oil, Eucalyptus Globulus Oil, Fennel (Foeniculum Vulgare) Oil, Gardenia Florida Oil, Geranium Maculatum Oil, Ginger (Zingiber Officinale) Oil, Gold of Pleasure (Camelina Sativa) Oil, Grapefruit (Citrus Grandis) Oil, Hops (Humulus Lupulus) Oil, Hypericum Perforatum Oil, Hyptis Suaveolens Oil, Indigo Bush (Dalea Spinosa) Oil, Jasmine (Jasminum Officinale) Oil, Juniperus Communis Oil, Juniperus Virginiana Oil, Labdanum (Cistus Labdaniferus) Oil, Laurel (Laurus Nobilis) Oil, Lavandin (Lavandula Hybrida) Oil, Lavender (Lavandula Angustifolia) Oil, Lemon (Citrus Medica Limonum) Oil, Lemongrass (Cymbopogon Schoenanthus) Oil, Leptospermum Scoparium Oil, Lime (Citrus Aurantifolia) Oil, Linden (Tilia Cordata) Oil, Litsea Cubeba Oil, Lovage (Levisticum Officinale) Oil, Mandarin Orange (Citrus Nobilis) Oil, Massoy Bark Oil, Matricaria (Chamomilla Recutita) Oil, Moroccan Chamomile Oil, Musk Rose (Rosa Moschata) Oil, Myrrh (Commiphora Myrrha) Oil, Myrtle (Myrtus Communis) Oil, Norway Spruce (Picea Excelsa) Oil, Nutmeg (Myristica Fragrans) Oil, Olax Dissitiflora Oil, Olibanum, Opoponax Oil, Orange (Citrus Aurantium Dulcis) Flower Oil, Orange (Citrus Aurantium Dulcis) Oil, Palmarosa (Cymbopogon Martini) Oil, Parsley (Carum Petroselinum) Seed Oil, Passionflower (Passiflora Incarnata) Oil, Patchouli (Pogcstemon Cablin) Oil, Pelargonium Graveolens Oil, Pennyroyal (Mentha Pulegium) Oil, Peppermint (Mentha Piperita) Oil, Pine (Pinus Palustris) Oil, Pine (Pinus Palustris) Tar Oil, Pine (Pinus Pinea) Kernel Oil, Pine (Pinus Pumiho) Oil, Pine (Pinus Sylvestris) Cone Oil, Rosemary (Rosmarinus Officinalis) Oil, Rose Oil, Rosewood (Aniba Rosseodora) Oil, Rue (Ruts Graveolens) Oil, Sage (Salvia Officinalis) Oil, Sambucus Nigra Oil, Sandalwood (Santalum Album) Oil, Sandarac (Callitris Quadrivalvis) Gum, Sassafras Officinale Oil, Sisymbrium Ino Oil, Spearmint (Mentha Viridis) Oil, Sweet Marjoram (Origanum Majorana) Oil, Sweet Violet (Viola Odorata) Oil, Tar Oil, Thuja Occidentalis Oil, Thyme (Thymus Vulgaris) Oil, Vetiveria Zizanoides Oil, Wild Mint (Mentha Arvensis) Oil, Ximenia Americana Oil, Yarrow (Achillea Millefolium) Oil, Ylang Yang (Cananga Odorata) Oil, or any combinations thereof.

Preferably, the one or more essential oils used in the present lotion composition is chamomile oil, lavendin oil, or any combination thereof.

The one or more essential oils are present in the lotion composition in an amount about 0.00001 wt. % to about 1 wt. %, based on the total weight of the composition. Preferably, the one or more essential oils are present in an amount about 0.00005 wt. % to about 0.1 wt. %, and more preferably about 0.0001 wt. % to about 0.015 wt. %., of the total weight of the composition.

The lotion composition of the present invention may include one or more emulsifiers. Suitable emulsifiers that can be used in the present invention include, for example, one or more alkoxylated fatty alcohols, alkylpolyglycosides, soaps, alkyl sulfates, monoalkyl and dialkyl phosphates, alkyl sulphonates, acyl isothionates, cetyl alcohol, sorbitans, stearic acid, or any combinations thereof. Preferred emulsifiers include cetyl alcohol, stearic acid, or any combinations thereof.

When used in the present composition, the emulsifier is about 1 wt. % to about 10 wt. % of the total weight of the composition. More preferably, emulsifier is present in an amount about 3 wt. % to about 8 wt. % of the total weight of the composition.

The present lotion composition may also include one or more stabilizers to stabilize the emulsion of the present composition. Suitable stabilizers include, for example, one or more alcohols, alkoxylated alcohols, fatty alcohols, glyceryl esters, such as, glyceryl stearate, gums, soaps, synthetic polymers, waxes, or any combinations thereof. Preferably, the one or more emulsion stabilizers are one or more glyceryl stearates. Preferably, the one or more glyceryl esters are one or more glyceryl stearates.

The one or more stabilizers are present in the lotion composition in an amount about 1 wt. % to about 10 wt. %. Preferably, the emulsion stabilizer is present in an amount about 2 wt. % to about 8 wt. %, and more preferably about 3 wt. % to about 5 wt. % of the total weight of the composition.

The present lotion composition may also include one or more emollients to condition the skin. Suitable emollients for use in the present lotion composition include, for example, one or more fats and oils, such as, coconut oil and cocoa butter, esters, such as, tricapryl citrate, or any combinations thereof. Preferably, the one or more emollients are coconut oil, cocoa butter, tricapryl citrate, or any combinations thereof.

The one or more emollients are present in the lotion composition in an amount about 0.01 wt. % to about 15 wt. %. Preferably, the one or emollients are present in an amount about 1 wt. % to about 10 wt. % and more preferably about 3 wt. % to about 6 wt. %.

The present lotion composition preferably has a solvent. Suitable solvents for use in the lotion composition include, for example, water, alcohol, polyhydric alcohol, glycerin, glycol, or any combinations thereof. Preferably, the solvent is water.

The solvent is present in the composition in an amount about 65 wt. % to about 90 wt. % based on the total weight of the composition. Preferably, the solvent is present in an amount about 75 wt. % to about 85 wt. %.

The composition can also include one or more additional components. Suitable additional components may include, for example, one or more humectants, skin conditioners, preservatives, chelating agents, pH adjusters, thickeners, surfactants, fragrances and/or perfumes, colorants, or any combinations thereof.

The composition may include one or more humectants. A humectant is a component that absorbs or retains moisture. Suitable humectants that can be used in the present composition include, for example, urea, pyroglutamic acid, amino acid, polyol or other compounds with hygroscopic properties, or any combinations thereof. Preferably, the one or more humectants are one or more polyols. Preferably, the one or more polyols are one or more sorbitols.

When present, the humectant is in an amount about 0.1 wt. % to about 3 wt. %. Preferably, the humectant is present in an amount about 0.5 wt. % to about 1.5 wt. %, of the total weight of the composition.

The lotion composition of the present invention may also have one or more skin conditioners. Suitable skin conditioners include, for example, one or more silicones, such as, dimethicone, aloe vera gel, or any combinations thereof.

When present, the skin conditioners are in an amount about 0.1 wt. % to about 5 wt. % of the total weight of the composition. Preferably, the skin conditioners are present in an amount about 0.5 wt. % to about 2 wt. %.

The composition may have one or more preservatives. Suitable preservatives for use in the present composition include, for example, one or more alkanols, EDTA salts, EDTA fatty acid conjugates, isothiazolinone, parabens such as methylparaben and propylparaben, propylene glycols, sorbates, urea derivatives such as diazolidinyl urea and imidazolidinyl urea, quaternary ammonium salts, such as, quaternium 15, or any combinations thereof. Preferably, the preservatives include imidazolidinyl urea, methylparaben, propylparaben, quaternium 15, or any combinations thereof.

When present, the preservatives are in an amount about 0.1 wt. % to about 1.5 wt. % of the total weight of the composition. Preferably, the preservatives are present in an amount about 0.3 wt. % to 1 wt. % of the total weight of the composition.

The composition may include one or more chelating agents to enhance the efficacy of the one or more preservatives. Suitable chelating agents may include, for example, ethylenediaminetetraacetic acid (EDTA), EDTA derivative, or any combinations thereof. Preferably, the one or more chelating agents is disodium EDTA.

The chelating agent is present in the lotion composition in an amount about 0.01 wt. % to about 0.2 wt. %. Preferably, chelating agent is present in an amount about 0.05 wt. % to about 0.15 wt. %, of the total weight of the composition.

The composition may include one or more pH adjusters. Suitable pH adjusters include, for example, one or more adipic acids, glycines, citric acids, calcium hydroxides, magnesium aluminometasilicates, triethanolamine, or any combinations thereof. The pH adjuster is added to maintain desired pH levels. Preferably, triethanolamine is used as the pH adjuster. When present, the pH adjuster is about 0.1 wt. % to about 2 wt. %, and preferably about 0.6 wt. % to about 1 wt. %, of the total weight of the composition.

The pH of the lotion composition of the present invention is about 4 to about 9. Preferably, the pH is about 6.5 to about 7.5.

The lotion composition of the present invention may include one or more thickeners. Suitable thickeners may include, for example, synthetic polymer, acrylates copolymer, acrylates crosspolymer, acrylic acid copolymer, adipic acid copolymer, polyethylene, polyvinyl acetate, polyvinyl alcohol, polyvinyl chloride, PVP/Eicosene copolymer, or any combinations thereof. Preferably, the thickener is an acrylates crosspolymer. Preferably, the acrylates crosspolymer is an acrylates C₁₀₋₃₀ alkyl/acrylate crosspolymer sold under the tradename Carbopol 1342® by Goodrich.

When present, the one or more thickeners are in an amount about 0.01 wt. % to about 0.1 wt. % of the total weight of the composition. Preferably, the one or more thickeners are present in an amount about 0.03 wt. % to about 0.07 wt. %.

The lotion composition may also include one or more surfactants. Suitable surfactants include, for example, anionic, nonionic, cationic, amphoteric, or any combinations thereof. Preferably the one or more surfactants are nonionic surfactants.

Suitable nonionic surfactants include, for example, one or more alkoxylated alcohols, ethoxylated alcohols, propoxylated alcohols, inter-dispersed ethoxylated-propoxylated alcohols, copolymers, fatty acids, alkyl phenols, polyglycosides, polyglucosides, n-alkylpyrrolidones, block copolymers, or any combinations thereof. Preferably, the nonionic surfactant is one or more alkoxylated alcohols. A preferred alkoxylated alcohol is PEG-75 lanolin.

When present, the one or more surfactants are in an amount about 0.2 wt. % to about 1 wt. %. Preferably, the one or more surfactants are present in an amount about 0.3 wt. % to about 0.7 wt. %.

The lotion composition of the present invention has a viscosity about 5,000 centipoise to about 50,000 centipoise. Preferably, the viscosity is about 12,000 centipoise to about 35,000 centipoise, and more preferably about 15,000 centipoise to about 30,000 centipoise.

The composition may include one or more perfumes and/or fragrances other than the one or more essential oils. Preferably, the one or more essential oils are supplied as components of a fragrance component, therefore eliminating the need for a separate fragrance and/or perfume component.

The following example represents a preferred composition of the present invention, as well as a preferred range for each ingredient listed.

### EXAMPLE 1

Lotion Composition

| | | |
|---|---|---|
| | Range | |
| Ingredient | (wt. %) | Purpose |

| | | |
|---|---|---|
| DI water | QS | solvent |
| Carbopol 1342 | 0.03-0.07 | thickener |
| Triethanolamine | 0.6-1 | pH adjuster |
| Disodium EDTA | 0.05-0.15 | chelating agent |
| Methylparaben | 0.1-0.4 | preservative |
| Imidazolidinyl urea | 0.1-0.3 | preservative |
| Sorbitol | 0.5-3 | humectant |
| Aloe vera gel | 0.1-1 | skin conditioning |
| | | agent |
| Tricapryl citrate | 1-4 | emollient |
| Coconut oil | 1-4 | emollient |
| Cocoa butter | 0.01-0.08 | emollient |
| Glyceryl stearate | 3-5 | emulsion stabilizer |
| Stearic acid | 2.5-4.5 | emulsifier |
| Cetyl alcohol | 1-3 | emulsifier |
| PEG-75 lanolin | 0.2-1 | surfactant |
| Dimethicone | 0.5-2 | skin conditioning |
| | | agent |
| Propylparaben | 0.07-0.13 | preservative |
| Hydrolactin 2500 | 0.01-0.5 | moisturizer/film |
| | | former |
| Quaternium 15 | 0.07-0.13 | preservative |
| Fragrance (3% | 0.2-0.4 | fragrance/essential |
| lavendin, 0.05% | | oil |
| chamomile) | | |

The lotion composition of the present invention is formulated as a stable emulsion. The emulsion can be an oil-in-water emulsion or a water-in-oil emulsion. Preferably, the lotion composition is a water-in-oil emulsion.

In the water phase of the emulsion, the present composition has one or more of the following components: milk protein compound, solvent, thickener, pH adjuster, chelating agent, skin conditioning agent, humectant, preservative, or any combinations thereof.

The oil phase includes one or more of the following components: essential oil, emollient, emulsifier, emulsion stabilizer, surfactant, skin conditioning agent, preservative, or any combinations thereof.

The present invention also includes a method for simultaneously moisturizing skin while calming and healing the user of the lotion through aromatherapy. The method comprises the step of applying to the skin a lotion composition having one or more milk protein compounds and one or more essential oils.

The foregoing specification is merely illustrative of the present invention and is not intended to limit the present invention to the disclosed embodiments. Variations and changes, which are obvious to one skilled in the art are intended to be within the scope and nature of the present invention.

What is claimed is:

1. A lotion composition comprising a milk protein, an essential oil, and an emulsifier.

2. The lotion composition of claim 1, wherein said milk protein is selected from the group consisting of hydrolyzed milk protein, hydrolyzed milk protein derivative, and any combinations thereof.

3. The lotion composition of claim 1, wherein said milk protein is present in an amount about 0.001 wt % to about 1 wt % based on the total weight of the composition.

4. The lotion composition of claim 1, wherein said milk protein is present in an amount about 0.01 wt % to about 0.5 wt % based on the total weight of the composition.

5. The lotion composition of claim 1, wherein said essential oil is selected from the group consisting of chamomile oil, lavendin oil, and any combinations thereof.

6. The lotion composition of claim 1, wherein said essential oil is present in an amount about 0.00001 wt % to about 1 wt % based on the total weight of the composition.

7. The lotion composition of claim 1, wherein said essential oil is present in an amount about 0.00005 wt. % to about 0.1 wt. % based on the total weight of the composition.

8. The lotion composition of claim 1, wherein said essential oil is present in an amount about 0.0001 wt. % to about 0.015 wt. % based on the total weight of the composition.

9. The lotion composition of claim 1, wherein said emulsifier is selected from the group consisting of alkoxylated fatty alcohol, alkylpolyglycoside, soap, alkyl sulfate, monoalkyl and dialkyl phosphate, alkyl sulphonate, acyl isothionate, cetyl alcohol, sorbitan, stearic acid, and any combinations thereof.

10. The lotion composition of claim 1, wherein said emulsifier is present in an amount about 1 wt. % to about 10 wt. % of the total weight of the composition.

11. The lotion composition of claim 1, wherein said emulsifier is about present in an amount 3 wt. % to about 8 wt. % of the total weight of the composition.

12. The lotion composition of claim 1, further comprising a stabilizer.

13. The lotion composition of claim 12, wherein said stabilizer is selected from the group consisting of alcohol, alkoxylated alcohol, fatty alcohol, glyceryl ester, gum, soap, synthetic polymer, wax, and any combinations thereof.

14. The lotion composition of claim 12, wherein said stabilizer is present in an amount about 1 wt % to about 10 wt % of the total weight of the composition.

15. The lotion composition of claim 12, wherein said stabilizer is present in an amount about 2 wt % to about 8 wt % of the total weight of the composition.

16. The lotion composition of claim 12, wherein said stabilizer is present in an amount about 3 wt % to about 5 wt % of the total weight of the composition.

17. The lotion composition of claim 1, further comprising an emollient.

18. The lotion composition of claim 17, wherein said emollient is selected from the group consisting of one or more fats, oils, esters, and any combinations thereof.

19. The lotion composition of claim 17, wherein said emollient is present in an amount 0.01 wt. % to about 15 wt. % of the total weight of the composition.

20. The lotion composition of claim 17, wherein said emollient is present in an amount 1 wt. % to about 10 wt. % of the total weight of the composition.

21. The lotion composition of claim 17, wherein said emollient is present in an amount 3 wt. % to about 6 wt. % of the total weight of the composition.

22. The lotion composition of claim 1, further comprising a solvent.

23. The lotion composition of claim 22, wherein said solvent selected from the group consisting of water, alcohol, polyhydric alcohol, glycerin, glycol, and any combinations thereof.

24. The lotion composition of claim 22, wherein said solvent is present in an amount about 65 wt. % to about 90 wt. % of the total weight of the composition.

25. The lotion composition of claim 22, wherein said solvent is present in an amount about 75 wt. % to about 85 wt. % of the total weight of the composition.

26. The lotion composition of claim 1, further comprising a humectant.

27. The lotion composition of claim 26, wherein said humectant is selected from the group consisting of urea, pyroglutamic acid, amino acid, polyol, and any combinations thereof.

28. The lotion composition of claim 26, wherein said humectant is present in an amount about 0.1 wt % to about 3 wt % of the total weight of the composition.

29. The lotion composition of claim 26, wherein said humectant is present in an amount about 0.5 wt % to about 1.5 wt % of the total weight of the composition.

30. The lotion composition of claim 1, further comprising a skin conditioner.

31. The lotion composition of claim 30, wherein said skin conditioner is selected from the group consisting of silicones, such as, dimethicone, aloe vera gel, and any combinations thereof.

32. The lotion composition of claim 30, wherein said skin conditioner is present in an amount about 0.1 wt % to about 5 wt % of the total weight of the composition.

33. The lotion composition of claim 30, wherein said skin conditioner is present in an amount about 0.5 wt % to about 2 wt % of the total weight of the composition.

34. The lotion composition of claim 1, further comprising a preservative.

35. The lotion composition of claim 34, wherein said preservative is selected from the group consisting of alkanol, EDTA salt, EDTA fatty acid conjugate, isothiazolinone, paraben, diazolidinyl urea, imidazolidinyl urea, quaternary ammonium salt, and any combinations thereof.

36. The lotion composition of claim 34, wherein said preservative is present in an amount about 0.1 wt. % to about 1.5 wt. % of the total weight of the composition.

37. The lotion composition of claim 34, wherein said preservative is present in an amount about 0.3 wt. % to about 1 wt. % of the total weight of the composition.

38. The lotion composition of claim 1, further comprising a chelating agent.

39. The lotion composition of claim 38, wherein said chelating agent is selected from the group consisting of ethylenediaminetetraacetic acid (EDTA), EDTA derivative, and any combinations thereof.

40. The lotion composition of claim 40, wherein said chelating agent is present in an amount 0.01 wt. % to about 0.2 wt. % based on the total weight of the composition.

41. The lotion composition of claim 1, further comprising a pH adjuster.

42. The lotion composition of claim 41, wherein said pH adjuster is selected from the group consisting of acidic acid, glycine, citric acid, calcium hydroxide, magnesium aluminometasilicate, triethanolamine, and any combinations thereof.

43. The lotion composition of claim 41, wherein said pH adjuster is present in an amount 0.1 wt. % to about 2 wt. % based on the total weight of the composition.

44. The lotion composition of claim 1, further comprising a thickener.

45. The lotion composition of claim 44, wherein said thickener is selected from the group consisting of synthetic polymer, acrylates copolymer, acrylates crosspolymer, acrylic acid copolymer, adipic acid copolymer, polyethylene, polyvinyl acetate, polyvinyl alcohol, polyvinyl chloride, PVP/Eicosene copolymer, and any combinations thereof.

46. The lotion composition of claim 44, wherein said thickener is present in an amount 0.01 wt. % to about 0.1 wt. % of the total weight of the composition.

47. The lotion composition of claim 1, further comprising a surfactant.

48. The lotion composition of claim 47, wherein said surfactant is selected from the group consisting of alkoxylated alcohol, ethoxylated alcohol, propoxylated alcohol, inter-dispersed ethoxylated-propoxylated alcohol, copolymer, fatty acid, alkyl phenol, polyglycoside, polyglucoside, n-alkylpyrrolidone, block copolymer, and any combinations thereof.

49. The lotion composition of claim 47, wherein said surfactant is present in an amount 0.2 wt. % to about 1 wt. % of the total weight of the composition.

50. A lotion composition comprising
a milk protein;
an essential oil;
and an emulsifier;
wherein said milk protein is present in an amount about 0.001 wt % to about 1 wt % of the total weight of the composition, and
wherein said essential oil is selected from the group consisting of
chamomile oil,
lavendin oil,
and any combinations thereof.

51. The lotion composition of claim 50, wherein said lotion composition viscosity is about 5,000 centipoise to about 50,000 centipoise.
